(19) United States
(12) Patent Application Publication
(10) Pub. No.: US 2007/0238893 A1
(43) Pub. Date: Oct. 11, 2007

(54) Asymmetric hydrogenation of acyl enamides

(76) Inventors: Lee Boulton, Cambridge (GB); Celine Praquin, Canterbury (GB)

Correspondence Address: KENYON & KENYON LLP, ONE BROADWAY, NEW YORK, NY, 10004, US

(21) Appl. No.: 11/593,936

(22) Filed: Nov. 6, 2006

Related U.S. Application Data
(60) Provisional application No. 60/733,744, filed on Nov. 4, 2005.
(60) Provisional application No. 60/737,094, filed on Nov. 15, 2005.

(51) Int. Cl. C07C 205/04

(52) U.S. Cl. 560/21

(57) ABSTRACT

The present invention relates to the asymmetric hydrogenation of acyl enamides for preparing carbamoyl acylamide indan derivatives, which are useful intermediates for the preparation of compounds used in the treatment of various CNS disorders.

### CROSS REFERENCE TO RELATED APPLICATIONS

The present application claims the benefit of the following U.S. Provisional Patent Applications No. 60/733,744, filed Nov. 4, 2005, and No. 60/737,094, filed Nov. 15, 2005. The contents of these applications are incorporated herein by reference.

### FIELD OF THE INVENTION

The present invention relates to the asymmetric hydrogenation of acyl enamides for preparing carbamoyl acylamide indan derivatives, which are useful intermediates for the preparation of compounds used in the treatment of various CNS disorders.

### BACKGROUND OF THE INVENTION

Carbamoyl acylamide indan derivatives are intermediates useful for preparing carbamoyl aminoindan derivatives such as for example Ladostigil. The chemical structure of a carbamoyl acylamide indan is:

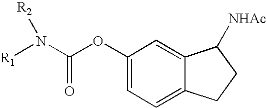

The carbamoyl aminoindan derivatives, which may be prepared from intermediates prepared by the present invention, have been shown to be effective in Alzheimer's disease. In Alzheimer's type dementia a common pathological feature is the lack of the neurotransmitter acetylcholine. This has led to the development of acetylcholine esterase inhibitors for use in the treatment of Alzheimer's disease.

Ladostigil is an example of such carbamoyl aminoindan derivative and is an active pharmaceutical ingredient which has shown to be effective in animal models of Alzheimer's disease. It also contains a (R)—N-propargyl aminoindan moiety which is a monoamine oxidase type B inhibitor. Ladostigil is disclosed in Weinstock, M. et al: J Neuronal Transm. (2000) [suppl]; 60: 157-169, Weinstock, M. et al: Development Research (2000); 50:216-222, Sterling J. et al: J. Med. Chem. 2002; 45:5260-5279, Weinstock M. et al: Psychopharmacology 2002; 160:318-324; and Yogev-Falach et al: FASEB J. 2002; Oct. 16(12):1674-1676. Ladostigil, N-ethyl-N-methylcarbamic acid (R)-2,3-dihydro-3-(2-propynylamino)-1H-inden-5-yl ester, has the following chemical structure:

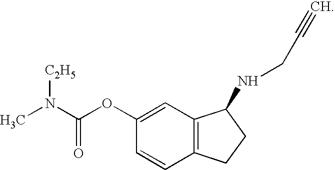

A method for preparing carbamoyl aminoindan derivatives is described in U.S. Pat. No. 6,303,650. The '650 patent describes the preparation of carbamoyl propargyl aminoindan derivatives by preparing a carbamoyl aminoindan from a hydroxy aminoindan and a carbamoylhalogenide. The carbamoyl aminioindan is reacted with an appropriate propargyl compound to prepare a carbamoyl propargyl aminoindan.

The methods of preparing pharmaceutically active carbamoyl aminoindan derivatives as described often results in racemic mixtures of the various enantiomers of the desired compounds. Typically, however, only one optically active enantiomer of the compound is pharmaceutically active. Therefore, there is a need to prepare optically active enantiomers of the carbamoyl aminoindan derivatives. Useful for the preparation of such compounds are stereospecific intermediates for processes to prepare an enantiomer of a carbamoyl aminoindan derivative.

Burk et al. (J. Org. Chem.; 1998; 63, 6084-6085) describe a three step process for asymmetric catalytic reductive amidation of ketones. The process contains a step using a stereospecific catalyst for the asymmetric hydrogenation of an alkaloyl enamide.

Bertand et al. (WO2005/082838) describe a process for the preparation of optically active substituted alpha-indanyl amide derivatives, which inlcudes the asymmetric hydrogenation of an enamide derivative.

The present invention is directed to the preparation of stereospecific enantiomers of acylamide indans, intermediates for the preparation of aminoindan derivatives, from carbamoyl substituted enamides. The stereospecific hydrogenation of enamides using an asymmetric transition metal catalyst is a complex reaction process. Consequently, various substitutions on the enamide substrate may affect the hydrogenation process differently, for example some substituents may even prevent the stereospecific hydrogenation of such substituted enamides. The present invention thus provides a process of asymmetric hydrogenation of carbamoyl enamides to provide optically active carbamoyl acylamide indans as appropriate intermediates for the synthesis of aminoindan derivatives, such as Ladostigil.

### SUMMARY OF THE INVENTION

The present invention provides an isolated enantiomer of a carbamoyl acylamide indan of formula I 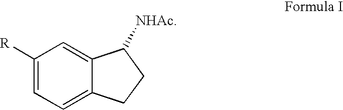
A preferred carbamoyl acylamide indan is ethylmethylcarbamoyl acylamide indan useful as an intermediate in the synthesis of Ladostigil.

In another aspect of the invention there is provided a method for preparing a carbamoyl acylamide indan compound of formula I 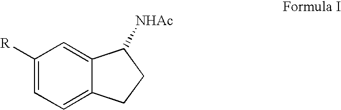
comprising the steps of,

a) providing an acyl enamide of formula II, and

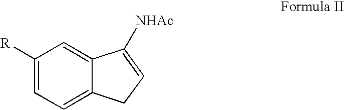

b) hydrogenating the acyl enamide of formula II in the presence of a catalyst to form the enantiomeric compound of formula I, wherein R is a carbamoyl represented by R₁R₂NCOO—, wherein R₁ and R₂ are each independently selected from hydrogen, a straight or branched chain C₁-C₆ alkyl group or a benzyl group. Preferably, the C₁-C₆ alkyl group is a C₁-C₄ alkyl group. More preferably R₁ is methyl. More preferably R₂ is ethyl. Most preferably, R₁ is methyl and R₂ is ethyl. The catalysts are preferably asymmetic transition metal catalyst, in particular the homogeneous chiral ligand transition metal precatalyst of the formula [L-M X]Y, wherein L is a chiral ligand, M is a transition metal, X is an organic moiety and Y is an anion.

In another aspect the present invention also provides a method of preparing a carbamoyl acylamide indan of formula I comprising the asymmetric hydrogenation of a carbamoyl acyl enamide of formula II in the presence of a catalyst, wherein the acyl enamide of formula II is prepared by a process comprising the steps of,

a) reacting 6-hydroxy indanone with a carbamoylhalogenide of formula III 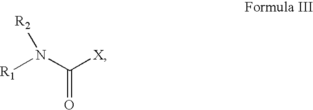
wherein X is a halogen, in a reaction mixture to form a carbamoyl indanone of formula IV,

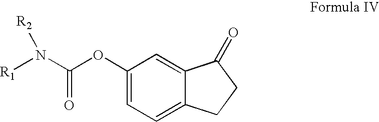

b) reacting the carbamoyl indanone of formula IV with a hydroxylamine in the presence of a base to form a carbamoyl oxime of formula V, and

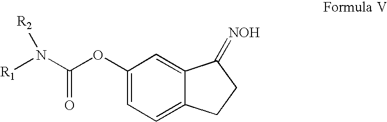

c) reducing the carbamoyl oxime of formula V to form the carbamoyl acyl enamide of formula II, wherein R₁ and R₂ are each independently a hydrogen, a straight or branched alkyl group or a benzyl group. Preferably, the reduction of the carbamoyl oxime of formula V is with iron metal in the presence of acetic anhydride in an organic solvent.

### DETAILED DESCRIPTION OF THE INVENTION

As used herein, the term precatalyst refers to a catalyst in a stable form which does not itself act as a catalyst but which will form an active catalyst in situ. Often the precatalyst of a chiral ligand transition metal catalyst as referred to in the present invention comprises the transition metal catalyst and an appropriate organic moiety which stabilizes the catalyst such as for example, cyclooctadiene (COD).

As used herein, the term;

BPE refers to 1,2-bis(substituted-phospholano)ethane and isomers;

DuPhos refers to bis(substitutes-phospholano)benzene.

In one embodiment, the present invention provides a method for preparing a carbamoyl acylamide indan compound of formula I 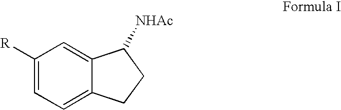
comprising the steps of,

a) providing an acyl enamide of formula II, and

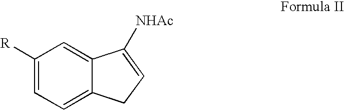

b) hydrogenating the acyl enamide of formula II in the presence of a catalyst to form the enantiomeric compound of formula I, wherein R is a carbamoyl represented by R₁R₂NCOO—, wherein R₁ and R₂ are each independently selected from hydrogen, a straight or branched chain C₁-C₆ alkyl group or a benzyl group. Preferably, the C₁-C₆ alkyl group is a C₁-C₄ alkyl group. More preferably R₁ is methyl. More preferably R₂ is ethyl. Most preferably, R₁ is methyl and R₂ is ethyl.

The catalysts are preferably asymmetic transition metal catalyst, in particular the homogeneous chiral ligand transition metal precatalyst of the formula [L-M X]Y, wherein L is a chiral ligand, preferably a chiral phosphine ligand, M is a transition metal, X is an organic moiety and Y is an anion.

The transition metal M of the catalyst used in the present invention is preferably selected from the group consisting of ruthenium (Ru), rhodium (Rh) and iridium (Ir). Most preferably the transition metal M is rhodium.

The anion Y in the catalyst used in the present invention is preferably selected from the groups consisting of ClO₄⁻, BF₄⁻, PF₆⁻, and SbF₆⁻. Most preferably the anion Y is BF₄⁻.

The organic moiety X may be an arene group having from 6 to 20 carbon atoms, preferably 6 to 12 carbon atoms, or an unsaturated organic group having from 2 to 20 carbon atoms, preferably from 2 to 10 carbon atoms, cyclic or not, selected from the group consisting of olefin, dienes having from 4 to 16 carbon atoms, preferably from 4 to 10 carbon atoms, and cyano. Preferably, the organic moiety is an arene such as benzene, para cymene, toluene, haexamethyl benzene and methoxybenzene or a diene such as 1,3-butadiene, 2,5-norbornadiene (NBD), 1,5-cyclooctadiene (COD) and cyclopentadiene. More preferably, the organic moiety is a diene such as 1,3-butadiene, 2,5-norbornadiene (NBD), 1,5-cyclooctadiene (COD) and cyclopentadiene, and most preferably cyclooctadiene (COD).

The chiral ligand L is selected from the group consisting of chiral diphosphine derivatives, chiral atropoisomeric diphosphine derivatives, chiral monodentate phosphoramidine derivatives, chiral biphospholane derivatives, chiral FerroTANE derivatives and chiral ferrocenyl phosphine derivatives. Preferably, the chiral ligand is a DuPhos, or BPE based ligand, more preferably the chiral ligand is selected from the group consisting of (R,R)-Me-DuPhos, (R,R)-Et-DuPhos, (R,R)-Me-BPE, (R,R)-Et-BPE, and (S,S)—Ph-BPE.

The chiral ligand transition metal catalyst can be prepared in situ or can be a preformed complex. Preferably, a preformed complex/precatalyst is used that is activated in situ. The precatalyst is preferably selected from the group consisting of [(R,R)-Me-DuPhos Rh COD]BF₄, [(R,R)-Et-DuPhos Rh COD]BF₄, [(R,R)-Me-BPE Rh COD]BF₄, [(R,R)-Et-BPE Rh COD]BF₄, and [(S,S)—Ph-BPE Rh COD]BF₄.

An acyl enamide of formula II is hydrogenated by the method of the present invention to form an enantiomer of an acylamide indan of formula I. To prepare the enantiomer of an acylamide indan the stereospecific hydrogenation of an acyl enamide is carried out in the presence of a chiral ligand transition metal catalyst in an organic solvent. Preferably, this hydrogenation is carried out by preparing a reaction mixture of an acyl enamide of formula II in an organic solvent. The reaction mixture is maintained at a particular reaction temperature and hydrogen (H₂) pressure in the presence of the chiral ligand transition metal catalyst.

The organic solvent used during the asymmetric hydrogenation is preferably selected from the group consisting of an ether such as tetrahydrofuran (THF), tetrahydropyran and diethyl ether, an aromatic hydrocarbon such as benzene and toluene, a halogenated hydrocarbon such as dichloromethane, and an alcohol such as methanol, ethanol or isopropanol. In a preferred embodiment of the invention the solvent used is an alcohol, preferably a C₁-C₄ alcohol, most preferably the solvent is methanol.

The asymmetric hydrogenation of the acyl enamide substrate in the method of the present invention is carried out where the acyl enamide substrate is present in the reaction mixture in an amount in excess to the amount of the catalyst. The molar ratio of the substrate (S), carbamoyl acyl enamide of formula (II), to the catalyst (C) used during the asymmetric hydrogenation is from S/C=50/1 to S/C=10000/1, preferably from S/C=100/1 to S/C=5000/1, more preferably from S/C=1000/1 to S/C=5000/1, most preferably at S/C=5000/1.

The hydrogen pressure used during the asymmetric hydrogenation is from about 0.5 to about 20 bar, preferably from about 0.5 to about 15 bar, more preferably from about 1 to about 10 bar, most preferably from about 4 to about 10 bar.

The temperature range used during the asymmetric hydrogenation is from about −20° C. to about 100° C., preferably from about 20° C. to about 100° C., more preferably from about 20° C. to about 60° C. and most preferably from about 40° C. to about 60° C. The temperature is maintained during the asymmetric hydrogenation of the present invention for a period of time in the range from about 10 min to about three days, preferably from about one hour to about three days, more preferably from about 1 hour to about 1 day and most preferably from about 4 hours to about 1 day.

Moreover, it is understood that any combination of the preferred catalyst, solvent, molar excess, hydrogen pressure and temperature as described above provides an asymmetric hydrogenation of an acyl enamide of formula II to form an enantiomeric compound of formula I as in the method of the present invention.

A preferred embodiment of the present invention can be represented by the following scheme I showing a method of asymmetric hydrogenation of a carbamoyl acyl enamide.

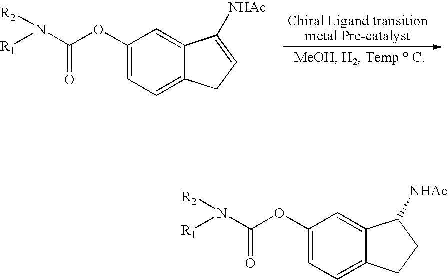

In another embodiment of the invention there is provided a method of preparing a carbamoyl acylamide indan of formula I comprising the hydrogenation of an carbamoyl acyl enamide of formula II in the presence of a catalyst, wherein the carbamoyl acyl enamide of formula II is prepared by a process comprising the steps of,

a) reacting 6-hydroxy indanone with a carbamoylhalogenide of formula III 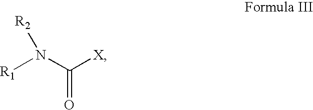
wherein X is a halogen, preferably chloride, in a reaction mixture to form a carbamoyl indanone of formula IV,

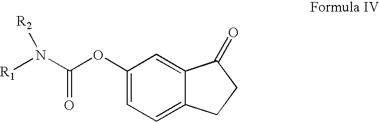

b) reacting the carbamoyl indanone of formula IV with a hydroxylamine, preferably hydroxylamine HCl, in the presence of a base to form a carbamoyl oxime of formula V, and

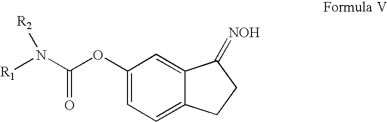

c) reducing the carbamoyl oxime of formula V to form the carbamoyl acyl enamide of formula II, wherein R₁ and R₂ are each independently a hydrogen, a straight or branched alkyl group or a benzyl group. Preferably, the reduction of the carbamoyl oxime of formula V is with a metal in the presence of acetic anhydride in an organic solvent.

The reaction process of preparing an carbamoyl enamide is carried out in a suitable organic solvent, preferably the organic solvent is dimethylformamide (DMF), a mixture of dimethylformamide and toluene, acetic acid, or a mixture of acetic acid and toluene, more preferably the organic solvent is dimethylformamide (DMF). In reacting 6-hydroxy indanone with a carbamoylhalogenide preferably a suitable base is added to the reaction mixture to form the carbamoyl indanone of formula IV. Preferably the base is an alkalimetal base, such as NaOH, KOH, Na₂CO₃, K₂CO₃, Li₂CO₃, or Cs₂CO₃. More preferably the base is an alkalimetal carbonate, most preferably the base is potassium carbonate (K₂CO₃). The carbamoylhalogenide is preferably a carbamoyl chloride of formula III, wherein R₁ and R₂ are independently a hydrogen, a straight or branched alkyl group or a benzyl group. In a preferred carbamoylhalogenide of formula III, R₁ is methyl and R₂ is ethyl, more preferably the carbamoylhalogenide is carbamoylchloride.

The carbamoyl indanone of formula IV is transformed to the carbamoyl oxime of formula V in the presence of hydroxylamine hydrochloride and a suitable base such as an alkali acetate. Preferably, sodium acetate. The transformation is carried out in a suitable organic solvent, preferably an alcohol, more preferably methanol. Further, this transformation can be carried out by adding the carbamoyl indanone of formula IV to a suspension of hydroxylamine hydrochloride and an alkali acetate in an organic solvent forming a reaction mixture. This suspension of hydroxylamine hydrochloride and alkali acetate in an organic solvent is preferably in a 1:1 molar ratio. Subsequently, the reaction mixture is agitated for a sufficient period to complete the transformation of the carbamoyl indanone of formula IV to a carbamoyl oxime of formula V. Preferably, the reaction mixture is stirred for about 1 to 4 hours, preferably for about 2 hours, at a temperature from about 20° C. to about 40° C., preferably at about room temperature. The carbamoyl oxime of formula V can be purified and isolated from the reaction mixture by any known method. Preferably, the carbamoyl oxime of formula V is obtained by concentrating the reaction mixture under reduced pressure and subsequently adding water to the formed reaction slurry. The reaction slurry is than agitated for a period of time sufficient to produce a precipitate, preferably from about 30 minutes to about 2 hours, more preferably for about 1 hour. The precipitate formed can be isolated through filtration.

Reduction of the carbamoyl oxime of formula V is preferably prepared using a metal in the presence of acetic anhydride. Preferably, the metal is Fe or Ru. In addition, the reaction temperature is preferably kept at a moderate temperature to avoid product decomposition. The reaction temperature is no more than about 75° C., preferably from about room temperature to about 75° C., most preferably the reaction temperature is about 75° C. Furthermore, acetic acid is preferably added to this reaction mixture to increase the reduction rate of the oxime. A preferred amount of acetic acid added to the reaction mixture is from about 2 equivalents per mol of the oxime to about 4 equivalents, more preferably about 3 equivalents. To improve reaction conditions and facilitate product isolation a cosolvent is added to the reaction mixture. This cosolvent is an organic solvent, preferably toluene or DMF.

A preferred embodiment of the present invention of a method of preparing a carbamoyl acyl enamide of formula II and the subsequent asymmetric hydrogenation thereof to form a carbamoyl acylamide of formula I can be represented by the following scheme II.

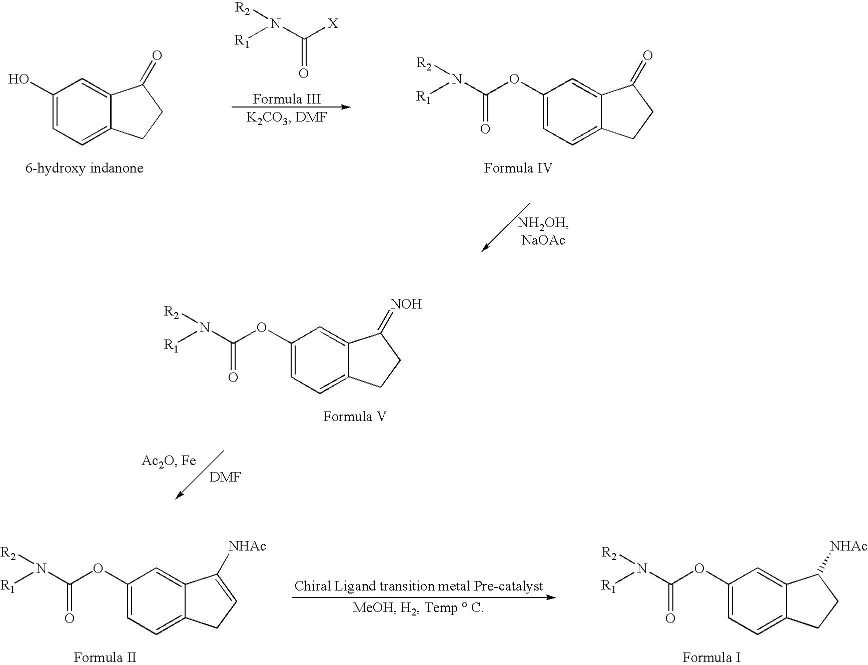

In another embodiment of the invention there is provided an isolated enantiomer of a carbamoyl acylamide indans of formula I. These stereospecific compounds are useful intermediates in preparing compounds used in the treatment of various CNS disorders. In particular, these carbamoyl acylamide indans are useful as intermediates in preparing carbamoyl aminoindan derivatives of formula VI: 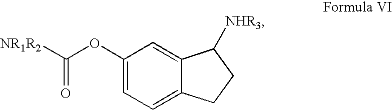
wherein R₁ and R₂ are defined as above and R₃ is a substituted or unsubstituted, straight or branched C₁-C₆ alkyl or heteroalkyl. A preferred carbamoyl acylamide indan of formula I, ethylmethylcarbamoyl acylamide indan, is useful as an intermediate in the synthesis of Lodastigil, an active pharmaceutical ingredient shown to be effective in animal models in the treatment of Alzheimer's disease.

Having described the invention with reference to certain preferred embodiments, other embodiments will become apparent to one skilled in the art from consideration of the specification. The disclosures of the prior art references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Example 1

### Asymmetric Hydrogenation in Preparing a Carbamoyl Acylamide Indan Enantiomer

A dimethylcarbamoyl acyl enamide was dissolved in methanol. Hydrogenation of the compound was performed in the presence of a rhodium precatalyst with the enamide in methanol forming a reaction mixture. The ratio of enamide:precatalyst (S/C) was 100:1 (S/C=100). The hydrogenation reaction mixture was maintained for a period of 5 to 12 hours at 40° C. and a hydrogen pressure of 5 to 10 bar. In Table 1 the relative amount of conversion and enantiomeric excess is shown for representative precatalyst used.

| | | Enantiomeric |
|---|---|---|
| Pre-catalyst | Conversion (%) | Excess (%) |
| [(R,R)-Me-DuPhos Rh COD]BF₄, | >98 | 97 |
| [(R,R)-Et-DuPhos Rh COD]BF₄, | >98 | 89 |
| [(R,R)-Me-BPE Rh COD]BF₄ | >98 | 98 |
| [(R,R)-Et-BPE Rh COD]BF₄ | >98 | 94 |
| [(S,S)-Ph-BPE Rh COD]BF₄ | >98 | 85 |

### Example 2

### Preparation of N-Acetyl-6-(N,N-dimethyl carbamate)-1-amino-indane

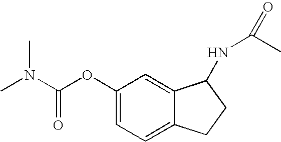

[(S,S)-Me-BPE Rh COD]BF₄ (2.1 mg, 0.0038 mmol) and N-(6-(N,N-dimethyl carbamate)-3H-inden-1-yl)acetamide (0.099 g, 0.38 mmol) were placed in a glass liner within a Baskerville 10 well autoclave and the vessel assembled. The vessel was pressurized to 10 bar with nitrogen and then the gas was released, this process was repeated a further three times. After the final vent, degassed methanol (3 mL) was introduced into the bomb. The vessel was then charged and vented three times with hydrogen to 10 bar. The vessel was heated to 40° C. (internal) whilst maintaining the pressure at 10 bar. After stirring overnight, the vessel was allowed to cool, the hydrogen pressure was released and the vessel was disassembled. The reaction mixture was concentrated under reduced pressure to afford a crude residue, which was analysed by ¹H NMR spectroscopy for conversion and chiral HPLC for enantioselectivity. ¹H NMR (400 MHz, d₆-DMSO) δ ppm 8.28 (1H, d, J 8, NH), 7.24 (1H, d, J8, Ar), 6.95 (1H, dd, J8 and 2, Ar), 6.91 (1H, d, J 1, Ar), 5.27 (1H, dt, J8 and 8, CH), 3.06 (3H, s, MeN), 2.96-2.89 [4H, m, MeN and CHH, incl. at 2.93 (3H, s, MeN)], 2.83-2.77 (1H, m, CHH), 2.44-2.40 (1H, m, CHH), 1.90 (3H, s, MeCO) and 1.87-1.80 (1H, m, CHH). The N-Acetyl-6-(N,N,-dimethyl carbamate-1-amino indane was obtained in a 98% ee (enantiomeric excess).

### Example 3

### Preparation of N-Acetyl-6-(N,N-methyl-ethyl carbamate)-1-amino-indane

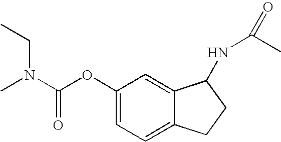

[(R,R)-MeBPE Rh COD]BF₄ (3.6 mg, 0.006 mmol) and N-(6-(N,N-methyl-ethyl carbamate)-3H-inden-1-yl)acetamide (1.23 g, 4.4 mmol) were placed in a glass liner within an 50 mL hydrogenation vessel and the vessel assembled. The vessel was pressurized to 5 bar with nitrogen and then the gas was released, this process was repeated a further three times. After the final vent, degassed methanol (10 mL) was introduced into the bomb. The vessel was then charged and vented three times with hydrogen to 5 bar. After stirring for 5 hours, the hydrogen pressure was released and the vessel was disassembled. The reaction mixture was concentrated under reduced pressure to afford the title compound as a brown powder (1.30 g, quant.(i.e. a yield of 100%)), which was analysed by HPLC for enantioselectivity. ¹H NMR (400 MHz, d₆-DMSO) δ ppm 8.29 (1H, d, J 8, NH), 7.24 (1H, d, J7, Ar), 6.95 (1H, d, J8, Ar), 6.91 (1H, s, Ar), 5.28 (1H, dt, J8 and 8, CH), 3.45-3.41 (Rotamer A, 1H, m, CH₂N), 3.34-3.31 (Rotamer B, 1H, m, CH₂N), 3.04 (Rotamer A, 1.5H, s, MeN), 2.91 (Rotamer B, 1.5H, s, MeN), 2.96-2.89 (1H, m, CHH), 2.83-2.75 (1H, m, CHH), 2.46-2.38 (1H, m, CHH), 1.90 (3H, s, MeCO), 1.85-1.80 (1H, m, CHH), 1.20 (Rotamer A, 1.5H, t, J 6, Me) and 1.12 (Rotamer B, 1.5H, t, J 6, Me). The N-Acetyl-6-(N,N-methyl-ethyl carbamate)-1-amino-indane was obtained in a 98% ee (enantiomeric excess).

### Example 4

### Preparation of Dimethyl-carbamic acid 3-oxo-indan-5-yl ester

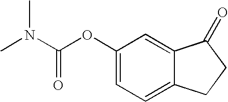

Dimethyl carbamyl chloride (7.7 mL, 83.3 mmol) was added dropwise to a stirred suspension of 6-hydroxy-1-indanone (10.290 g, 69.4 mmol) and potassium carbonate (12.48 g, 90.3 mmol) in DMF (50 mL) at 0° C. (external) over a period of 30 minutes. One hour after the addition was complete the cold bath was removed and the reaction was allowed to warm slowly to room temperature over 2 hours. The reaction mixture was diluted with methyl tert-butyl ether (50 mL) and water (100 mL) and the resultant solid was collected by filtration and washed with water (50 mL) and then methyl tert-butyl ether (50 mL). The collected material was dried under vacuum overnight. The crude product was purified by solvent slurry in methyl tert-butyl ether (50 mL) before being collected by filtration, washed with additional methyl tert-butyl ether (20 mL) and dried to yield the desired compound (14.877 g, 98%). ¹H NMR (400 MHz, CDCl₃) δ ppm 7.47-7.45 (2H, m, Ar), 7.36 (1H, dd, J8 and 2, Ar), 3.14-3.11 [5H, m, OCCH₂ and Me, incl. at 3.11 (3H, s, Me)], 3.02 (3H, s, Me) and 2.74-2.71 (2H, m, OCCH₂CH₂).

### Example 5

### Preparation of 6-(N,N-Dimethylcarbamate)-1-indanone oxime

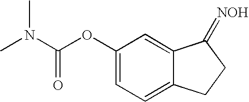

Sodium acetate (4.64 g, 56.5 mmol) was added to a stirred suspension of hydroxylamine hydrochloride (3.84 g, 55.2 mmol) in methanol (75 mL). The resulting slurry was stirred at room temperature for 20 minutes before 6-(N,N-dimethylcarbamate)-1-indanone (9.95 g, 45.3 mmol) was added portionwise over 45 mins. After stirring for 2 hours the reaction mixture was concentrated under reduced pressure, water (65 mL) was added to the residue and the resulting slurry was stirred for 1 hour. The precipitate was collected by filtration and washed with water to yield the desired compound as a pale yellow powder (9.75 g, 92%). ¹H NMR (400 MHz, d₆-DMSO) δ ppm 10.99 (1H, s, OH), 7.37 (1H, d, J8, Ar), 7.25 (1H, d, J2, Ar), 7.10 (1H, dd, J 8 and 2, Ar), 3.07 (3H, s, MeN), 3.00 (2H, t, J 6, CH₂), 2.94 (3H, s, MeN) and 2.86-2.83 (2H, m, CH₂).

### Example 6

### Preparation of N-(6-(N,N-Dimethyl carbamate)-3H-inden-1-yl)acetamide

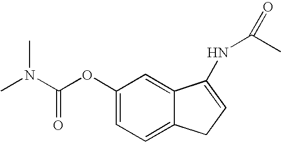

Acetic anhydride (12.0 mL, 127.7 mmol) was added slowly to a stirred solution of 6-(N,N-dimethylcarbamate)-indanone oxime (9.65 g, 41.1 mmol) in a mixture of toluene (70 mL) and DMF (30 mL) containing iron (2.67 g, 47.8 mmol). The solution was slowly warmed up to 50° C. over 30 minutes. (Upon reaching 45° C., the temperature rose quickly to 78° C. and then returned to 50° C.). After stirring 2 hours at 50° C., TLC analysis indicated that the reaction had not gone to completion. (TLC, eluant: 50% toluene-ethyl acetate, R_{f}: oxime: 0.42; acetamide: 0.17).The reaction mixture was warmed up to 55° C. After stirring 1 hour at this temperature the reaction appeared to have gone to completion. The reaction mixture was allowed to cool to room temperature before being filtered through a pad of celite. Dichloromethane (2×50 mL) was used to wash the solid materials. The liquors were washed with saturated aqueous sodium hydrogen carbonate solution (2×80 mL), dried (MgSO₄), filtered. Silica (21 g) was added to the liquors and the slurry was stirred for 30 minutes then filtered. The liquors were concentrated under reduced pressure to afford a black solid. Purification by flash column chromatography using 50% toluene-ethyl acetate as eluant yielded the desired compound as a brown powder (5.62 g, 52%) ¹H NMR (400 MHz, d₆-DMSO) δ ppm 9.76 (1H, s, NH), 7.61 (1H, d, J2, Ar), 7.46 (1H, d, J8, Ar), 6.98 (1H, dd, J8 and 2, Ar), 6.83 (1H, t, J2, CH═C), 3.39 (2H, d, J2, CH₂), 3.11 (3H, s, MeN), 2.96 (3H, s, MeN) and 2.14 (3H, s, MeCO).

### Example 7

### N-(6-(N,N-Methyl-ethyl carbamate)-3H-inden-1-yl)acetamide

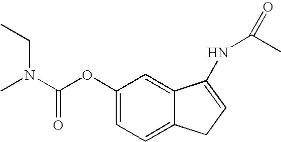

Acetic anhydride (11 mL, 117 mmol) was added slowly to a stirred solution of 6-(N,N-methyl-ethylcarbamate)-1-indanone oxime (9.08 g, 36.5 mmol) in a mixture of toluene (75 mL) and DMF (35 mL) containing iron (3.06 g, 54.8 mmol). The solution was slowly warmed up to 60° C. over 30 minutes. (Upon reaching 45° C., the temperature rose quickly to 83° C. but returned quickly to 60° C., when the heating source was removed). After stirring at 60° C. for 2 hours, the reaction mixture was cooled to room temperature before being filtered through a pad of celite. Dichloromethane (2×50 mL) was used to wash the solid material. The liquors were washed with saturated aqueous sodium hydrogen carbonate solution (2×100 mL), dried (MgSO₄), filtered then concentrated under reduced pressure to afford the impure N-(6-(N,N-Methyl-ethyl carbamate)-3H-inden-1-yl)acetamide as a black solid. Purification by flash column chromatography using 40% ethyl acetate-toluene as eluant afforded a beige solid. This material was slurried in ethyl acetate (75 mL) to give N-(6-(N,N-Methyl-ethyl carbamate)-3H-inden-1-yl)acetamide as an off-white powder (5.66 g, 56%). ¹H NMR (400 MHz, d₆-DMSO) δ ppm 9.77 (1H, s, NH), 7.61 (1H, d, J 1, Ar), 7.46 (1H, d, J8, Ar), 6.98 (1H, dd, J8 and 2, Ar), 6.83 (1H, s, CH═C), 3.52-3.46 (rotamer A, 1H, m, CH₂N), 3.40 (2H, d, J 2, CH₂), 3.37-3.34 (rotamer B, 1H, m, CH₂N), 3.09 (rotamer A, 1.5H, s, MeN), 2.95 (rotamer B, 1.5H, s, MeN), 2.14 (3H, s, MeCO), 1.25 (rotamer A, 1.5H, t, J 7, Me) and 1.15 (rotamer B, 1.5H, t, J 7, Me).

### Example 8

### 6-(N,N-Methyl-ethylcarbamate)-1-indanone oxime

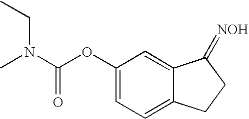

Sodium acetate (4.13 g, 50.3 mmol) was added to a stirred suspension of hydroxylamine hydrochloride (3.43 g, 49.3 mmol) in methanol (50 mL). The resulting slurry was stirred at room temperature for 30 minutes before 6-(N,N-methyl-ethylcarbamate)-1-indanone (9.22 g, 39.5 mmol) was added portionwise over 45 mins. After stirring for two hours, the reaction mixture was concentrated under reduced pressure, water (40 mL) was added to the residue and the resulting slurry was stirred for 1 hour. The precipitate was collected by filtration and washed with water to afford the 6-(N,N-Methyl-ethylcarbamate)-1-indanone oxime as a pale yellow powder (9.46 g, 96%). ¹H NMR (400 MHz, d₆-DMSO) δ ppm: 10.98 (1H, s, OH), 7.38 (1H, d, J8, Ar), 7.24 (1H, s, Ar), 7.10 (1H, dd, J8 and 2, Ar), 3.45 (rotamer A, 1H, q, J 7, CH₂N), 3.34 (rotamer B, 1H, m, CH₂N), 3.05 (rotamer A, 1.5H, s, MeN), 3.01 (2H, t, J6, CH₂), 2.93 (rotamer B, 1.5H, s, MeN), 2.86-2.80 (2H, m, CH₂), 1.22 (rotamer A, 1.5H, t, J 7, Me) and 1.13 (rotamer B, 1.5H, t, J 7, Me).

1. A method of preparing a carbamoyl acylamide indan compound of formula I 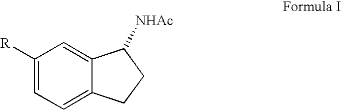
comprising the steps of,
a) providing an acyl enamide of formula II, and 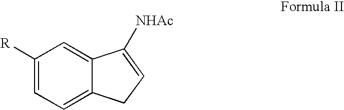
b) hydrogenating the acyl enamide of formula II in the presence of a catalyst to form the enantiomeric compound of formula I, wherein R is a carbamoyl represented by R₁R₂NCOO—, wherein R₁ and R₂ are each independently selected from hydrogen, a straight or branched chain C₁-C₆ alkyl group or a benzyl group.

2. The method of claim 1, wherein R₁ is methyl.

3. The method of claim 1, wherein R₂ is ethyl.

4. The method of claim 1, wherein R₁ is methyl and R₂ is ethyl.

5. The method of claim 1, wherein the catalyst is an asymmetric transition metal catalyst.

6. The method of claim 5, wherein the transition metal M of the catalyst is selected from the group consisting of ruthenium (Ru), rhodium (Rh) and iridium (Ir).

7. The method of claim 6, wherein the transition metal M of the catalyst is rhodium.

8. The method of claim 5, wherein the asymmetric transition metal catalyst is in the form of a homogeneous chiral ligand transition metal precatalyst of the formula [L−M X]Y, wherein L is a chiral ligand, M is a transition metal, X is an organic moiety and Y is an anion.

9. The method of claim 8, wherein the anion Y is selected from the groups consisting of ClO₄⁻, BF₄⁻, PF₆⁻, and SbF₆⁻.

10. The method of claim 9, wherein the anion Y is BF₄⁻.

11. The method of claim 8, wherein the organic moiety X is an arene group having from 6 to 20 carbon atoms or an unsaturated cyclic or acyclic organic group, selected from the group consisting of olefin, diene and cyano.

12. The method of claim 11, wherein the organic moiety is a diene selected from the group consisting of 1,3-butadiene, 2,5-norbornadiene, 1,5-cyclooctadiene (COD) and cyclopentadiene.

13. The method of claim 12, wherein the organic moiety is cyclooctadiene (COD).

14. The method of claim 8, wherein the chiral ligand L is selected from the group consisting of chiral diphosphine derivatives, chiral atropoisomeric diphosphine derivatives, chiral monodentate phosphoramidine derivatives, chiral biphospholane derivatives, chiral FerroTANE derivatives and chiral ferrocenyl phosphine derivatives.

15. The method of claim 14, wherein the chiral ligand L is selected from the group consisting of (R,R)-Me-DuPhos, (R,R)-Et-DuPhos, (R,R)-Me-BPE, (R,R)-Et-BPE, and (S,S)—Ph-BPE.

16. The method of claim 1, wherein the catalyst is activated in situ using a precatalyst selected from the group consisting of [(R,R)-Me-DuPhos Rh COD]BF₄, [(R,R)-Et-DuPhos Rh COD]BF₄, [(R,R)-Me-BPE Rh COD]BF₄, [(R,R)-Et-BPE Rh COD]BF₄, and [(S,S)—Ph-BPE Rh COD]BF₄.

17. The method of claim 1, wherein the hydrogenation is carried out in an organic solvent selected from the group consisting of an ether, an aromatic hydrocarbon, a halogenated hydrocarbon, and an alcohol.

18. The method of claim 17, wherein the organic solvent is selected from the group consisting of tetrahydrofuran, tetrahydropyran, diethylether, benzene, toluene, dichloromethane, methanol, ethanol and isopropanol.

19. The method of claim 18, wherein the organic solvent is methanol.

20. The method of claim 1, wherein the hydrogenation is carried out with the acyl enamide substrate present in the reaction mixture in an amount in excess to the amount of the catalyst, the molar ratio of the carbamoyl acyl enamide of formula (II) to the optically active chiral ligand transition metal catalyst in the range from 50/1 to 10000/1.

21. The method of claim 20, wherein the molar ratio is from 100/1 to 5000/1.

22. The method of claim 21, wherein the molar ratio is from 1000/1 to 5000/1.

23. The method of claim 1, wherein the hydrogenation is carried out under a hydrogen pressure from about 0.5 to about 20 bar.

24. The method of claim 23, wherein the hydrogen pressure is from about 1 to about 10 bar.

25. The method of claim 24, wherein the hydrogen pressure is from about 4 to about 10 bar.

26. The method of claim 1, wherein the hydrogenation is carried out at a temperature from about −20° C. to about 100° C. for a period of time of about 10 minutes to about three days.

27. The method of claim 26, wherein the temperature is from about 20° C. to about 100° C.

28. The method of claim 27, wherein the temperature is from about 40° C. to about 60° C.

29. The method of claim 26, wherein the period of time is from about 1 hour to about 1 day.

30. The method of claim 29, wherein the time period is from about 4 hours to about 1 day.

31. The method of claim 1, wherein the acyl enamide of formula II is prepared by a process comprising the steps of,
a) reacting 6-hydroxy indanone with a carbamoylhalogenide of formula III 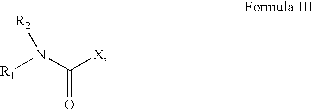
wherein X is a halogen, in a reaction mixture to form a carbamoyl indanone of formula IV, 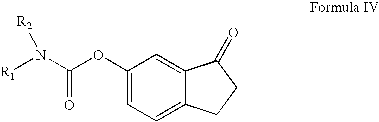
b) reacting the carbamoyl indanone of formula IV with a hydroxylamine in the presence of a base to form a carbamoyl oxime of formula V, and 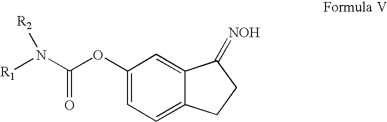
c) reducing the carbamoyl oxime of formula V to form the carbamoyl acyl enamide of formula II, wherein R₁ and R₂ are each independently a hydrogen, a straight or branched alkyl group or a benzyl group.

32. The method of claim 31, wherein the reduction of the carbamoyl oxime of formula V is with metal in the presence of acetic anhydride in an organic solvent.

33. The method of claim 32, wherein the metal is selected from the group consisting of Fe and Ru.

34. The method of claim 33, wherein the organic solvent is dimethylformamide.

35. The method of claim 31, wherein to the reaction mixture of step a) a base is added.

36. The method of claim 35, wherein the base is an alkalimetal carbonate.

38. The method of claim 37, wherein the alkalimetal carbonate is potassium carbonate.

38. The method of claim 31, wherein the carbamoylhalogenide of formula III is carbamoylchloride.

39. The method of claim 31, wherein R₁ is methyl and R₂ is ethyl in the carbamoylhalogenide of formula III.

40. The method of claim 31, wherein the base in step b) is an alkali acetate.

41. The method of claim 40, wherein the alkali acetate is sodium acetate.

42. The method of claim 31, wherein step b) is carried out in a reaction mixture with an organic solvent.

43. The method of claim 42, wherein the organic solvent is methanol.

44. The method of claim 42, wherein the reaction mixture is a 1:1 molar suspension of the combination of hydroxylamine and a base and the organic solvent.

45. The method of claim 42, wherein the reaction mixture is stirred for a period of about 1 to about 4 hours at a temperature of about 20° C. to about 40° C.

46. The method of claim 45, wherein the period is about 2 hours.

47. The method of claim 45, wherein the temperature is at about room temperature.

48. The method of claim 31, wherein the carbamoyl oxime of formula V is purified and isolated.

49. The method of claim 31, wherein the reduction in step c) is carried out at a temperature of not more than 75° C.

50. The method of claim 49, wherein in step c) about 2 equivalents to about 4 equivalents of acetic acid is added to the reaction mixture per mol of the carbamoyl oxime of formula V.

51. The method of claim 49, wherein in step c) an organic co-solvent is to the reaction mixture.

52. The method of claim 51, wherein the organic co-solvent is toluene.

53. An isolated enantiomer of a carbamoyl acylamide indan of formula I 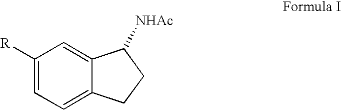
wherein R is a carbamoyl represented by R₁R₂NCOO—, wherein R₁ and R₂ are each independently selected from hydrogen, a straight or branched chain alkyl group or a benzyl group.

54. The isolated enantiomer of a carbamoyl acylamide indan of claim 53, wherein R₁ is methyl.

55. The isolated enantiomer of a carbamoyl acylamide indan of claim 53, wherein R₂ is ethyl.

57. The isolated enantiomer of a carbamoyl acylamide indan of claim 53, wherein R₁ is methyl and R₂ is ethyl.

58. A method of preparing a carbamoyl amino indan of formula VI 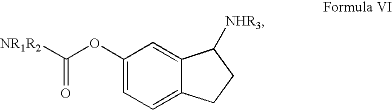
wherein R₁ and R₂ are each independently selected from hydrogen, a straight or branched chain alkyl group or a benzyl group, and R₃ is a substituted or unsubstituted, straight or branched C₁-C₆ alkyl or heteroalkyl from the isolated enantiomer of a carbamoyl acylamide indan of claim 53.

59. The method of claim 58, wherein the carbamoyl amino indan of formula VI is Ladostigil.
